(19) United States
(12) Patent Application Publication
(10) Pub. No.: US 2009/0132034 A1
(43) Pub. Date: May 21, 2009

(54) Bioprosthetic Valve Clip and Handle

(76) Inventors: Fernando Riveron, Wausau, WI (US); Donald Grover, Rothschild, WI (US); Fred Lane, Mosinee, WI (US)

Correspondence Address: LANE PATENTS LLC, 100 NORTH 72ND AVE., SUITE 107, WAUSAU, WI, 54401, US

(21) Appl. No.: 12/272,514

(22) Filed: Nov. 17, 2008

Related U.S. Application Data
(60) Provisional application No. 60/988,296, filed on Nov. 15, 2007.

(51) Int. Cl. A61F 2/24

(52) U.S. Cl. 623/2.11

(57) ABSTRACT

An improved implantation holder apparatus and system for holding bioprosthetic aortic valves for insertion into a heart. The handle system interacts with implantation holder and the valve in such a way that commissure support struts may be retracted by simply rotating the handle which allows the surgeon more room to work in tight quarters to complete the implantation process. Included is a handle system, which can be attached and detached, for securing the holder during application of the parachute stitching and moving the valve into position. Once the valve is in position, the handle may be retained in place, or removed for greater ease of access to the aortic cavity.

### BACKGROUND OF THE INVENTION

1. Field of the Invention

The present invention relates to the field of valvular prostheses, and, more particularly, to a bioprosthetic heart valve holding tool and system for implantation of aortic valves which retracts the struts during implantation and is minimally intrusive.

2. Description of the Related Art

The relevant art of interest describes various devices for implanting an aortic valve, in particular.

Many surgeons look to streamline the process of valve replacement. The process is quite invasive on the patient. The procedure for implantation is risky with the risk being increased the longer the procedure takes. The surgeon also views every point of the procedure as a possible point of error. The surgeon is continually looking to remove steps, minimize time, and eliminate possible points of error. In addition, access to the aorta by the surgeon requires working in tight quarters in best of conditions allowing little room to maneuver. Therefore, the surgeon requires a minimally invasive methodology, which allows the procedure to be completed with a least number of meaningful steps.

U.S. Pat. No. 7,189,258 B2 issued to Johnson et al Mar. 13, 2007; discloses a current valve and holder used widely in the art. It discloses a methodology built upon a thread and reel apparatus. This methodology, as a broad approach, dates back to at least 1979. This system has several drawbacks. Firstly; reeling of the spool, without also rotating the heart valve, requires that an opposing member be placed to keep the valve from rotating. This is accomplished by running a series of legs between the commissure posts and securing them with a suture. The suture at the bottom of the leg then acts as an anti-rotation member which places strain on that portion of the stent and accompanying material, which is not ideally desired. Further; many surgeons find in practice that their knots, required to suture the prosthetic in place, catch in the leg stent union due partially to the tight working quarters at the base. This is a potentially dangerous result, as loosely, poorly, or improperly tied sutures can lead to separation or partial separation of the stent and further lead to paravalvular leakage. In addition, the commissure support strut is also reeled in by a suture which is placed into the tissue at the apex of the strut, thus pulling on the prosthetic to retract the strut. This stress to the prosthetic is deemed undesirable. When the surgeon is finished with the process of implantation of the prosthetic, and in order to remove the holder, the surgeon is required to enter the cavity with a knife to remove the suture. This further risks the patient for being poked or cut with a knife, scalpel, or other sharp cutting device.

UK patent number GB 2 108 393 issued to Extracorporeal Medical Specialties Inc. Published 18 May 1983 and filed in 1979, was the first to disclose the approach upon which the Johnson patent is based.

U.S. Pat. Nos. 5,476,510 and 5,716,401 a child patent issued to Eberhard et al. Dec. 19, 1995 and Feb. 10, 1998 respectively disclose two inventions. First is a high profile valve holder with a line and reel type strut synch. Second is a push structure spread across three levels of strut attachment with cleated detents. The requirement of three levels of attachment makes this apparatus higher profile than is ideally required. The present invention, without this limitation, is much more elegant with a lower profile and all located on one plane.

U.S. Pat. No. 4,865,600 issued to Carpentier et al. Sep. 12, 1989; and assigned to Baxter International Inc. discloses a heart valve holder which can be mounted on the bottom of the valve and rotates to reel in sutures which pull in the struts. This invention is designed for mitral valve work and should not be relevant to any of the present disclosure. It is included to show the prevalence in the art of reel type structures.

U.S. Pat. No. 6,019,790 issued to Holmberg et al. Feb. 1, 2000; and assigned to St. Jude Medical, Inc., discloses a valve holder including a set of jaws which can be pinched or pivoted to close on the structure. It is a high profile, which goes against the objective of the present invention.

### SUMMARY OF THE INVENTION

Summary

The present invention relates to an implantation holder and system for holding a bioprothetic aortic valve for insertion into a heart. The handle system interacts with implantation holder and the valve in such a way that commissure support struts may be retracted by simply rotating the handle which allows the surgeon more room to work in tight quarters to complete the implantation process. Included is a handle system, which can be attached and detached, for securing the holder during application of the parachute stitching and moving the valve into position. Once the valve is in position, the handle may be retained in place, or removed for greater ease of access to the aortic cavity. Once the valve is fully sutured into place the handle may be easily reattached to the holder with an automatic cutting system to cut the sutures and allow the holder to be more easily retracted from the aorta.

It is therefore an object of the invention to incorporate a system comprising a bioprosthetic valve holder for implanting which allows retraction of commissure support struts.

It is another object of the invention to provide retraction of commissure support struts without unduly tensioning the suture for attaching the holder to the stent.

It is another object of the invention to allow rotational retraction of the commissure support struts without torquing of the stent relative to the handle or implant site.

It is another object of the invention to provide a retracting means for the support strut without a need for a series of legs being attached in the valley between the struts of the prosthesis.

It is another object of the invention that the retraction of the commissure support struts is easily reversible.

It is another object of the invention that a number of intermediate positions of the commissure support strut positions are available.

It is another object of the invention that the holder has a low vertical profile to maximize a surgeon's access to the aorta.

It is another object of the invention to allow the handle to be attached and detached as will during the procedure.

It is another object of the invention to allow a means for cutting an removing sutures which does not require the surgeon to insert a knife into the aortic cavity to remove the sutures which attach the holder to the prosthetic.

These and other advantages of the present invention will become readily apparent upon review of the following specification and drawings.

### BRIEF DESCRIPTION OF THE DRAWINGS

A complete understanding of the present invention may be obtained by reference to the accompanying drawings, when considered in conjunction with the subsequent, detailed description, in which:

FIG. 1 is an exploded view of a tricuspid valve bioprosthetic with an implantation holder;

FIG. 2 is a perspective view of an implantation holder in a closed position;

FIG. 3 is a perspective view of an implantation holder connected with a valve prosthesis in an open position;

FIG. 4a, b, c are respectively top perspective, bottom plan, and bottom perspective views of a cam knob for an implant holder;

FIG. 5a, b, c, d are respectively bottom, top perspective, bottom perspective, and a detail of one aspect (B) of a bottom perspective views of a prosthesis holder;

FIG. 6a, b, c are respectively top perspective, top, and bottom perspective views of the guide for an implant holder;

FIG. 7a, b are bottom views of a prosthesis holder showing comparative positions for open and closed respectively;

FIG. 8a, b are bottom views of cam knob coupled with a prosthesis holder showing the interaction between the cam and the holder for open and closed positions respectively;

FIG. 9a, b are bottom views of a guide, coupled with a cam knob and prosthesis holder showing interaction between these parts for the open and closed positions respectively;

FIG. 10a, b are side perspective and front partial cut away views respectively of a handle member holding an implantation holder with a valve prosthesis.

### DETAILED DESCRIPTION OF THE DRAWINGS

Typically the heart valves that occur in nature are either bicuspid, which comprise two lobes, or valves for regulating blood flow or, more commonly, tricuspid which comprise three lobes or valves. While the typical replacement bioprosthetic valve is modeled after a tricuspid system, those skilled in the art will note that this particular system and apparatus can be anticipated for a plurality of lobes.

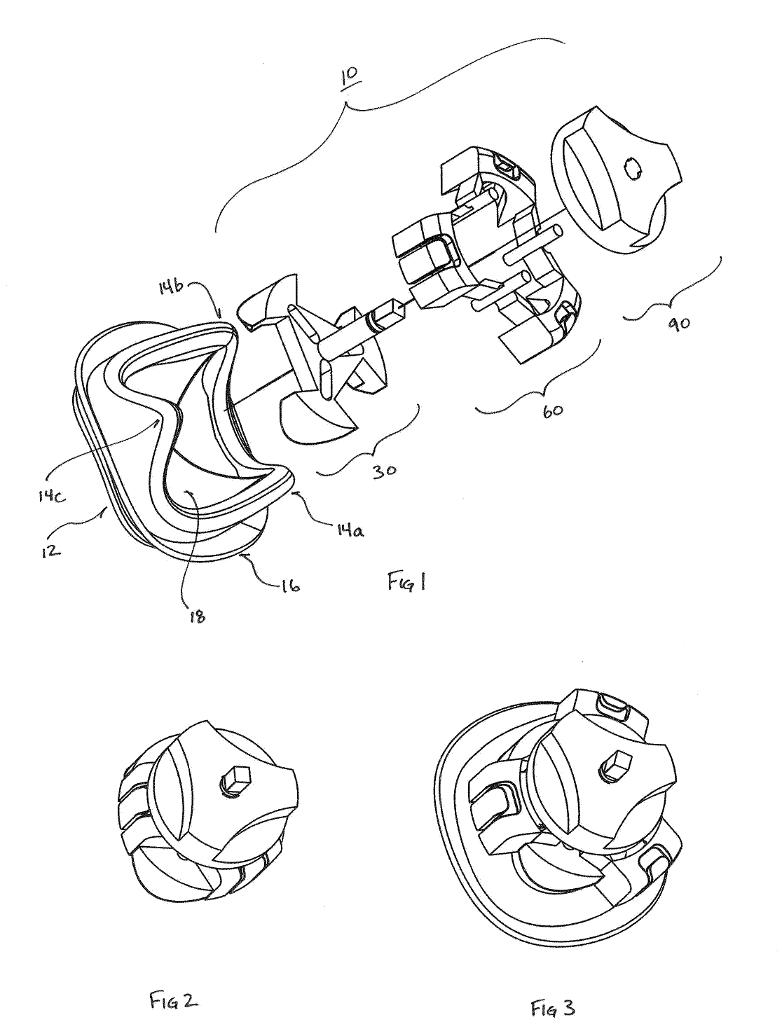

FIG. 1 shows and exploded view of the implantation holder 10 comprising its component parts with a valve prosthesis 12. A typical valve prosthesis 12 is typically comprised of three porcine supple valves 20, but may be of any suitable material of biologic or synthetic origin, such as urethane based polymers, which exhibits the qualities of a human heart valve material. The supple valves 20 are typically mounted to each of a commissure support strut 14a, b, c such that a one way valve is formed allowing minimal regurgitation between heart contractions. The valve prosthesis 12 further comprises a base 16 for forming the stent or opening for blood flow and can further comprise an attachment ring 18 for attaching, usually by sewing, and sealing the valve prosthesis 12 to the aortic wall.

The implantation holder 10 can further be broken down into several components comprising a guide 30, a prosthesis holder 60 and a cam knob 90 with their component elements and functions.

At least one significant advantage of the implantation holder 10 is shown in the comparison between FIG. 2 which is the implantation holder 10 in a closed position, and the implantation holder 10 in an open position as shown in FIG. 3. FIG. 3 further shows the valve prosthesis 12, which is not shown in FIG. 2. The closed position comprises a significant reduction in radial area of the commissure support strut 14, which allows the surgeon open access to the base 16.

In order to more fully appreciate the advantages of the current system, a more thorough coverage of the implantation holder 10 is required starting with the prosthesis holder 60 shown in FIGS. 5a through 5d. The prosthesis holder 6o is comprised of a plurality of receiving facets 62a, b, c which conform to and cradle the corresponding commissure support strut 14a, b,c. The receiving facets 62a, b, c are designed such that much of the stress related to retaining sutures, which can be applied in conjunction with the attachment location 78, is relieved during the transition from the open position to the closed position. This may be enhanced by the several retaining faces 66a, b, c optionally having a cleat 70 which contacts the face of the commissure support strut 14a, b, c preferably below any tissue area which may be present. The cleat 70 need not be obtrusive and can be formed in a number of embodiments; a cleat, a series of cones, or the like, such that the holding capability of the prosthesis holder 6o is increased.

The strut interface 64 can be formed such that the corresponding commissure support struts 14a, b, c are mirrored for greater stability.

Further the receiving facets 62a, b, c are in communication with one another and with the guide pins 76 through a plurality of coupling members 72. As the guide pins 76 are drawn into a tighter radius as denoted by the closed position, denoted in FIG. 2, the corresponding receiving facets 62a, b, c are also drawn in decreasing the diameter of the distal end of the commissure support strut 14a, b, c.

The closing of the prosthesis holder 6o is realized by operation of a cam knob 90 which communicates with the prosthesis holder 60. The cam knob 90 is comprised of a rotation grip 92, which in this instance is in the form of three curvilinear facets, but may take the form of a knurled knob or other suitable form sufficient for the surgeon to easily rotate the cam knob 90 with a gloved hand. A series of cam races 100, corresponding to the number of guide pins 76, are formed on the bottom face 96 of the cam knob 90 for coupling with the guide pins 76 for opening and closing the prosthesis holder 60 by rotating the cam knob 90 in either a clock-wise or counter clock-wise direction relative to the axis of the handle member 120. Further a series of click stops 102 may be placed along the length of the cam races 100 at predetermined intervals in order to allow a number of intermediate positions between fully opened and fully closed.

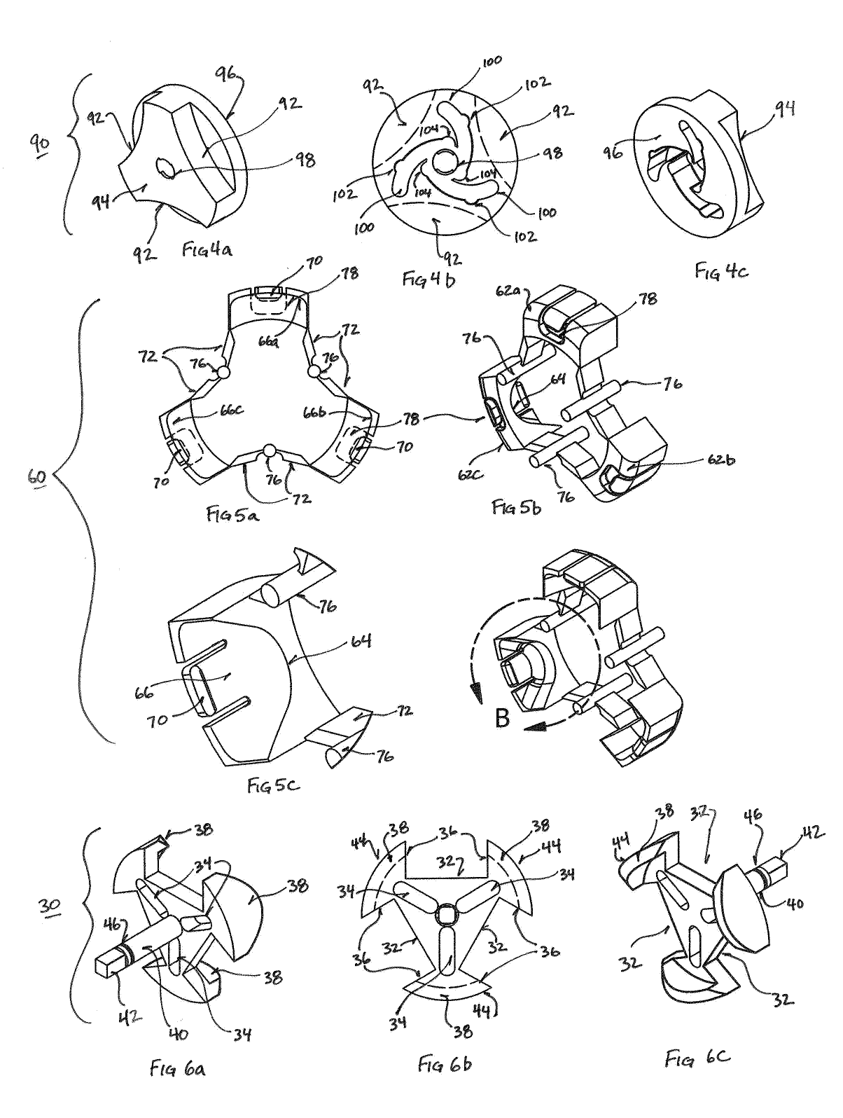

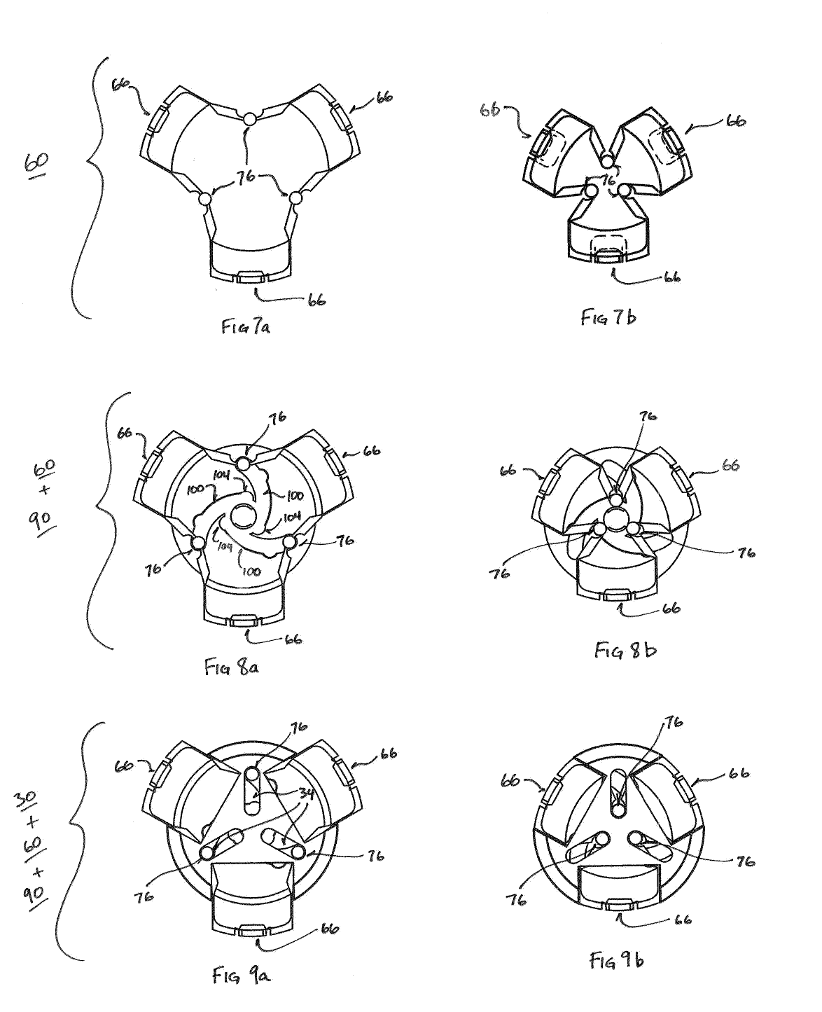

Anti-rotation and interface with the handle member 120 can be facilitated with the guide 30 shown in FIGS. 6a, b, and c. The guide 30 has a series of retraction slots 32 for guiding the receiving facets 62 during the opening and closing operations. The anti-rotation guide 34 receives the torque relative to the handle member 120 and the cam knob 90 as the cam knob 90 is rotated to move between the open and closed positions. This relieves stress to the tip of the strut from the method of sutures shown in the prior art. Further extending orthogonally from the guide 30 is an assembly post 40. As can be seen from FIG. 1, the assembly post 40 extends through the prosthesis holder 60 and the thread hole 98 of the cam knob 90 to anchor the assembled piece. Further the assembly post 40 can typically have one or more attachment key 46 to secure the handle member 120. A post interface 42, typically in the form of a faceted interface, may be located at the distal end of the assembly post 40 to provide a further rotation interface from the handle. In an alternate embodiment, the guide 30 can further comprise a series of prosthesis guards 38 extending over the opening formed between the commissure support strut 14 to further protect the delicate attachment ring 18. A feature of these prosthesis guards 38 is that they are designed with rounded edges and sloping slightly inward in such a way not to catch knots during suturing.

A typical example of the interrelationship of the component pieces for opening and closing of the commissure support strut 14a, b, c can be seen by visualizing FIGS. 7a, 7b, 8a, 8b, 9a, and 9b in concert. Figures with the “a” designation refer to the open position, and Figures with the “b” designation refer to the closed position.

FIGS. 7a, and b show the motion of the prosthesis holder 60 as the guide pins 76 are retracted and are coupled through the coupling members 72 with the retaining faces 66 which is in turn attached to the commissure support strut 14 (not shown in these Figures).

The tops of the guide pins 76 extend into the cam races 100 such that during rotation of the cam knob 90, the guide pins 76 move inwardly as shown in FIGS. 8a and 8b.

FIGS. 9a and 9b show the prosthesis holder 60 and cam knob 90 coupled with the guide 30. The guide 30 can be coupled with an anti-rotation guide 34 which, as can be seen by one skilled in the art, acts as an anti-rotation factor keeping any stresses formed between the twisting motion desired to retract the retaining faces 66a, b, c from being received in the corresponding commissure support strut 14a, b, c. In the present embodiment it is preferable that the outside of the retaining faces 66 be substantially the same diameter as the diameter formed by the perimeter 44 of the guide 30.

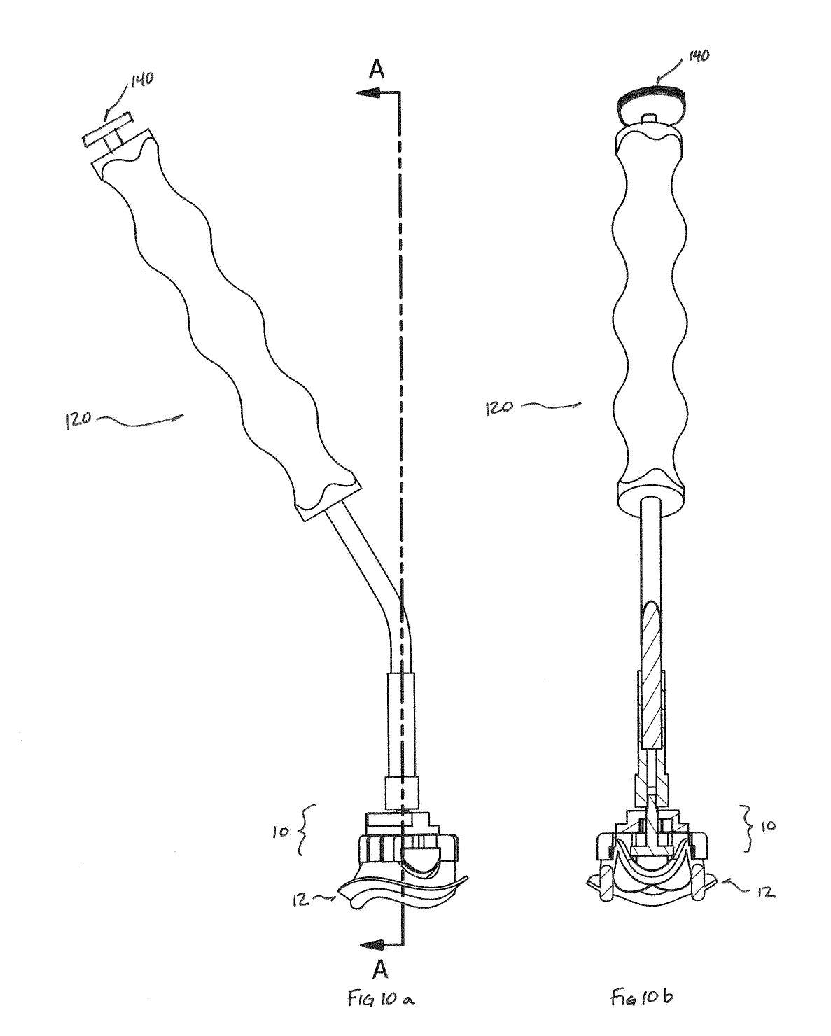

The implantation holder 10 with valve prosthesis 12 are positioned into place by a handle member 120, as is shown in FIGS. 10a and 10b. The handle member 120 may have an attachment interface 124, typically of the quick release type, being actuated by a quick release actuator 140, typically in the form of a button located at the distal end of the gripping surface 138. Further the post interface 42 forms a means for interfacing between the handle member 120 and the cam knob 90 such that when the cam knob is rotated, the handle and corresponding implantation holder 10 are relatively stationary.

### Conclusion, Ramifications, and Scope

Although the present invention had been described in detail, those skilled in the art will understand that various changes, substitutions, and alterations herein may be made without departing from the spirit and scope of the invention in its broadest form.

Having thus described the invention, what is desired to be protected by Letters Patent is presented in the subsequently appended claims.

What is claimed is:

1. A heart valve clip and handle system for inserting a bioprosthetic heart valve into the heart comprising:
a valve prosthesis comprising a plurality of commissure support struts extending orthogonally from the base of said valve prosthesis;
a prosthesis holder having a plurality of receiving facets being matched with, and removably connected to, said commissure support struts;
a cam knob being in communication with said receiving facets and having means for retracting said receiving facets which are coupled with said support struts;
a guide for coordinating said means for retracting being integrated with said cam knob and said prosthesis holder, and
a handle member being attached to said guide.

2. The heart valve clip and handle system in accordance with claim 1, wherein said guide comprises an anti-rotation guide.

3. The heart valve clip and handle system in accordance with claim 1, wherein said guide comprises a prosthesis guard.

4. The heart valve clip and handle system in accordance with claim 1, wherein said cam knob comprises means for retracting having a plurality of intermediate positions.

5. The heart valve clip and handle system in accordance with claim 1, wherein said cam knob comprises means for retracting which is reversable.

6. The heart valve clip and handle system in accordance with claim 1, wherein said cam knob comprises a rotation grip.

7. The heart valve clip and handle system in accordance with claim 1, wherein said handle member is detachable from said guide.

8. The heart valve clip and handle system in accordance with claim 7, wherein said handle member is reattchable to said guide.
